(19) United States
(12) Patent Application Publication
(10) Pub. No.: US 2009/0196852 A1
(43) Pub. Date: Aug. 6, 2009

(54) COMPOSITIONS AND METHODS FOR DIAGNOSING AND TREATING IMMUNE DISORDERS

(76) Inventor: D. Tobin Watkinson, Rancho Santa Fe, CA (US)

Correspondence Address: WILSON SONSINI GOODRICH & ROSATI, 650 PAGE MILL ROAD, PALO ALTO, CA, 94304-1050, US

(21) Appl. No.: 12/365,859

(22) Filed: Feb. 4, 2009

Related U.S. Application Data
(60) Provisional application No. 61/025,912, filed on Feb. 4, 2008.
(60) Provisional application No. 61/076,571, filed on Jun. 27, 2008.

(51) Int. Cl. A61K 38/21; C40B 30/04

(52) U.S. Cl. 424/85.4; 506/9

(57) ABSTRACT

The present invention is a method of detection to identify antigen reactivity between infective agents, e.g., bacteria, viruses, parasites, bacteriophages, spirochetes, microbes and common foods, beverages, inhalants, chemical preparations, perfumes, fragrances, pollens, animal proteins and dander. As a result of the specific identification of the pathogenic agent/antigen reaction, a vaccine can be constructed to eradicate such allergic reaction. The test platform represents a series of impregnated test modules containing sample libraries of infective materials which have potential cross reactivity with a human subject's biological fluid sample and a reagent when needed. Sequential retesting or manual biopanning for the positive reactions; allergen/bacteria, bacteria/virus, result in the underlying antigen/pathogenic agent reaction. Once such causative infective agent relationship is identified there are several options for vaccine preparation or treatment to reduce or eradicate the identified causative pathogenic agent.

### CROSS REFERENCE TO RELATED APPLICATIONS

This application claims the benefit of U.S. Provisional Application No. 61/025,912, entitled, “Compositions and Methods for Diagnosing and Treating Allergies,” filed Feb. 4, 2008, and U.S. Provisional Application No. 61/076,571, entitled “Compositions and Methods for Diagnosing and Treating Immune Disorders,” filed Jun. 27, 2008, both of which are incorporated herein by reference in its entirety.

### BACKGROUND OF THE INVENTION

The immune system in vertebrates provides a defense mechanism against foreign agents, including foreign macromolecules or infecting microorganisms. The immune system utilizes a common recognition system for both foreign macromolecules (e.g., proteins, polysaccharides or nucleic acids) or microbes (e.g. viruses or bacteria) through specific binding of the proteins of the host immune system to specific sites on the foreign agent, or antigen, surface, known as antigenic determinants.

The adaptive immune system, which is composed of specialized systemic cells, including T-cells, act to prevent pathogenic infestation. The adaptive immune system provides the ability to recognize and remember specific pathogens, and to initiate and generate an immune response to the pathogen. The system is adaptable to its host through the use of hypermutation and recombination processes, that allow the generation of a large number of different antigen receptors, which are then uniquely expressed on individual lymphocytes. Lymphocytes, including B-cells and T-cell lymphocytes play a large role in the immune response system. B-cells play a major role in the humoral immune response, while T-cells are intimately involved with cell-mediated immune responses, together with the helper T cell responses, Th1 and Th2.

The Th1 response, which responds to bacteria, virus, some parasites, some metals and vaccines, is characterized by the production of interferon-gamma, which activates the bactericidal activities of macrophages, and induces B-cells to make coating antibodies, leading to cell-mediated immunity. The Th2 response, which responds largely to allergies, mold, yeast, fungus, foods and some parasites and metals, is characterized by the release of interleukin 4 (IL4), which results in the activiation of B-cells to make neutralizing antibodies, leading to humoral immunity. Defects in the immune system may result or worsen symptoms in a variety of disorders, including chronic illness, such as chronic fatigue, fibromyalgia, Lyme's disease, autoimmune disorders, skin conditions, hair loss and asthma. Other conditions that may have a link to defects or weaknesses in the immune system include polycystic liver disease, polycystic kidney disease, autism, cancer and mental illness.

An allergy is a hypersensitive reaction to antigens, which in similar amounts and circumstances are harmless in other individuals. An allergic response to foreign agents is thought to develop when an individual's natural immune response is disturbed (atopy).

Our rapidly changing world environment has lead to allergy reaching monumental proportions and now affecting one out of every two people in the developed world since its discovery less than two hundred years. Allergy within the past two decades has increased largely among young people.

Allergy can affect many body systems, including sinus, skin, digestive, lungs, genital urinary, nervous system and can range from minor symptoms to life threatening emergencies and even death. Allergy can also serve as the catalyst for multiple infective diseases. Conversely, a disease state can serve as a catalyst for allergy.

Multiple theories have emerged to explain the exponential rise in the number of allergic and auto immune diseases. One of the more popular explanations centers on the growth of allergies and autoimmunity within developed countries. This theory points to the increased emphasis upon hygiene as a potential cause, postulating that the present allergic epidemic is partially due to changes in life style. The lack of exposure to significant infective agents prohibits those in more industrialized nations to go without the many opportunities in which to exercise the immune response that would result in active and complete antibody formation.

In addition industrialized countries with significant technological advances bring with them greater stresses upon the body which can result in the dampening of the immune response from an imbalance of the autonomic nervous system, i.e. the “flight or fight” response. Excessive sympathetic nervous system dominance and deficiency of the parasympathetic modulating affect results in a more unstable immune response. The unheard of emergence of allergies and autoimmune diseases in undeveloped countries may follow differences not only in life style but also environmental factors, including diet, disease management, water, hygiene, breast feeding, refrigeration, chemical use, farming and excess electronic exposure. The apparent difference in the percent of individuals suffering from allergies in industrialized countries all point to the potential role of these factors in the functioning of the immune system.

The environmental factors of industrialized countries may result in one or any number of immune response defects, resulting in mutations of proteinacious materials, amino acid sequences mismatching and pattern recognition errors. Any one of these errant processes can mimic infective elements and alter receptor sites, which then misinterpret the normal T lymphocyte response causing faulty immune activity. As it pertains to the immune system's need for exacting discrimination between self and non-self molecules, including foods, chemicals, beverages, environmental substances, fragrances, bacteria, viruses, spirochetes, cancers and infective microbes interaction at any receptor, synapses, cytokine, protein replication and other physiological systems, potential error in innate order becomes threatened.

The mechanism for immediate hypersensitive allergic reaction is presently understood to begin when an allergen is presented to a naive T lymphocyte. The T cell differentiates to a T helper cell (Th2) which through cytokine influence causes the B lymphocyte to secrete IgE immunoglobulin calling for mast cells, eosinophils and basophiles. Immediate hypersensitive allergy thus results in the increased production of IgE. The IgE is tested as a clinical indicator of the presence of immediate hypersensitivity (allergy) to either inhalants, consumables or contact.

Although current allergy theories point to environmental or other allergens providing the basis of an allergic reaction, one alternative theory traces atopy to a defect in the processing of allergens by the immune system. For example, peer reviewed medical literature report instances where allergy has its basis in cross-reactivity between otherwise unrelated proteins. The basis for such cross-reactivity of proteins of different organisms is thought to be due to the similarity of the epitope of the individual proteins which otherwise are unrelated. In one such case dog dander was cross reacting with human prostate specific antigen. Further examples of cross reactivity have also been shown between Candida, human tissue and food. In still other studies there have been examples of allergic reactions specific to both bacteria and to virus resulting in immunoglobulin evidence of such reactivity.

Numerous researchers have looked at mechanisms of action ranging from partial amino acid sequence matches to molecular mimicry to receptor site abnormalities. No study has yet given a definitive answer as to cause, although some investigators have offered further evidence that the allergenic immune reaction can improperly read vital substances, chemicals, environmental elements and even self as a faulty immune opportunity.

Within traditional medicine, an allergy diagnosis is made by skin prick or Ig blood tests (e.g. RAST testing for measurement of IgE) to identify the offending, allergic substances. As for alleviating the manifestations of allergic reactions, treatment has focused upon avoidance, desensitization, anti histamines and steroids and is largely symptomatic. The early diagnosis, treatment, and prevention of allergy have made very little progress in the last twenty years. There are presently several new approaches but none that have addressed the cause specifically through blocking receptors, altering genes, or novel methods of addressing symptoms.

### SUMMARY OF THE INVENTION

The present invention relates in part to the diagnosis and treatment of disorders related to the immune system, including allergies, chronic illness, chronic fatigue, fibromyalgia, Lyme's Disease, autoimmune disorders, skin conditions, hair loss, cancer, asthma, polycystic liver disease, polycystic kidney disease, autism and mental illness, by combining known testing methodologies, such as RAST or other tests detecting and/or quantifying the presence of immunoglobulins or other immunomodulatory molecules with complementary diagnostic tests to treat underlying causes related to disorders of the immune system, including allergic reactions. The underlying cause may include bacterial or other pathogenic or microbial infection resulting from incomplete processing by the immune system. Although not limited to this particular concept, it is believed through the research and studies disclosed herein that nucleoproteins, glycoprotein patterning, lipoprotein patterning and structural sequences of the environmental allergens mimic the infective agents underlying the allergic reaction, which in turn perpetuates the faulty response related to disorders of the immune system, including allergic reactions. Also contemplated within the present embodiments disclosed herein are methods of treatment of individuals suffering from disorders related to the immune system, with vaccines designed and developed with the information obtained from the diagnostic methods disclosed herein.

Accordingly, one embodiment disclosed herein are methods for identifying correlative relationships between antigenic agents, e.g. environmental, common foods, beverages, inhalants, chemical preparations, perfumes, fragrances, pollens, animal proteins and dander, and pathogenic agents, e.g. microbial agents, including bacteria, viruses, parasites, bacteriophages, spirochetes and other microbes, and environmental allergens, to determine the underlying cause related to disorders of the immune system, including allergic reactions. In some embodiments, the methods disclosed herein are used for determining underlying pathogen agents that may induce an adverse reaction, including allergic reactions with antigen(s) traditionally found responsible (e.g. dust mites, pollen, etc.) for the adverse reactions, including allergic reactions. The test platform represents a series of impregnated test modules containing sample libraries of infective material which have potential cross reactivity with a human subject's biological fluid sample, and a reagent when needed. Sequential testing or manual biopanning for positive reactions, e.g. allergen/bacteria, bacteria/virus, may result in the underlying allergenic/infective agent reaction. Such tests may determine through an iterative process, for example, a correlative relationship between antigens, including environmental allergen(s), and the underlying infectious agent, and provide information for later treatment of the underlying infectious pathway.

In addition to the correlative tests provided, also dislosed herein is a method for testing a mammalian patient to determine the underlying pathogen responsible for the adverse reaction related to disorders of the immune system (e.g. dust mites, pollen, etc.), and to correlate the antigenic(s) information with the underlying infectious agent that may be responsible for the adverse reaction, including allergic reactions. Such tests would incorporate results of the correlative studies that may determine the relationship between the antigen(s), including potential allergens, and underlying pathogenic or infectious agents. Correlative information may also be obtained through patient questionnaires regarding potential or known allergens that have been previously established in said patient.

Another embodiment of the present invention is a means for determining the underlying infectious agent that may be responsible for an adverse reaction related to an immune disorder, including but not limited to an allergic reaction. The underlying infectious agent may be tested directly from a patient's biological sample, for example, identification through molecular amplification reactions of a DNA fingerprint of the underlying infectious agent, or through, for example, acupuncture-based and kinesiology assays, including energetic testing, digital response technique (DRT), electrochemical detection of pathogens, electroacupuncture, galvanic skin response (GSR) and acupuncture point testing, including two-point muscle testing.

A method of treating a patient in need of such treatment is also disclosed. This method includes the steps of: 1) diagnosing the traditional antigen, e.g. allergen, responsible; 2) determining the correlative underlying infectious agent; and 3) treating said patient with a medicament or system that may reduce or eliminate the infectious agent from the patient. Conventional treatment with medicaments of microbial agents are contemplated. Such medicaments may include, but are not limited to, antibiotics, including pencillins, cephalosporins, tetracyclines, erythromycin, beta-lactams, sulfa drugs, including sulfonamides, and antifungal antibiotics, aminoglycosides, ansamycins, carbacepham, carbapenems, macrolides, quinolones, small molecule inhibitors polypeptides, anti-viral agents, including nucleoside analogues and interferon therapies, such as acyclovir, gancyclovir, zidovudine, lamivudine, ribavirin, amantidine and protease inhibitors. Alternatively, phage therapy, interleukin-interferon manipulation, or any other means of reducing or eliminating the underlying infectious agent or correcting or amelioration the related disorder in the immune system, may be used to reduce or eliminate the underlying microbial infective agent

Also contemplated herein is a method of formulating and treating a patient with a vaccine to lessen or eliminate an adverse reaction, such as an allergic reaction, wherein the vaccine is constructed from information obtained through the diagnostic methods disclosed above. The vaccine may be synthesized through any conventional means. Alternatively, the vaccine may be synthesized through bioelectric replication technology.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The methods and compositions of the embodiments disclosed herein are of use in the diagnosis and treatment of adverse reactions related to disorders of the immune system. The practices employed herein, unless otherwise indicated, include conventional techniques for determining adverse reactions to antigens, including environmental allergens. For example, allergy testing to detect and/or quantify reactive immunoglobulins from an individual patient may be employed with the embodiments disclosed herein. Such testing may include RAST (radioallergosorbent test), PRIST, skin prick testing or any other means of measuring immunoglobulins, including IgE, IgA, IgG or IgM. One exemplary method of detecting immunogenicity to a defined environmental allergen is the RAST test. The RAST test measures the immunogenic reaction to a specific allergen. For example, the allergen of interest, e.g. ragweed pollen or animal dander, is bound to a solid surface. A biological sample, such as blood, is added to the bound allergen, wherein only IgE antibody specific to the bound allergen reacts in the test sample. After the initial incubation, non-specific IgE antibody and other proteins are removed by washing. A labeled antibody specific to IgE antibodies is then added to detect the bound IgE antibodies to the solid platform. Phadia's ImmunoCAP® (Phadia, Uppsala Sweden) specific IgE blood test is one commercial example of a RAST test specific to IgE antibody detection. Other assays for use in conjunction with the methods disclosed herein include biometric tests, including heart rate variability tests, bioimpedance analysis, hormonal assays, including cortisol quantification and qualitative assays, as well as decreased secretory IgA markers, food allergies and specific biomarkers for various diseases, including but not limited to Crohn's disease, type I diabetes, multiple sclerosis, parasites and other diseases. Other tests may include energetic evaluations of patients. Decreased energy production is often accompanied by abonormalities of the immune system, which may result in an inability to maintain an immune response under static or stressed conditions.

Additionally, techniques for the identification of reactivity between identified antigens, for example allergens, and multiple infective agents will also be employed, including the sequential use of one or all of the following: electrophoresis, immunoblotting, PCR (polymerase chain reaction), ELISA (enzyme-linked immunosorbent assay), radioimmunoassay, immunofluorescence, galvanic skin response (GSR) testing, bio-resonant matching, electromagnetic testing for similarity or like techniques with or without reagents, which serve to open the diagnostic window for specific DNA or RNA identification.

Other biological or immunological assays may also be employed in conjunction with the identification of allergens and pathogens accompanying an abnormality of the immune system, including tests to measure autoimmune disease markers (anti-DNA, anti-nuclear antibody, anti-SSA, anti-SSB, anti-B polypeptide, anti-Jol, anti-Scl 70), immune system function panels (TH-1/TH-2 markers, related cytokines and growth factors), neuroimmunologic disorder markers (myelin basic protein, myelin associated glycoprotein, myelin oligodendrocyte glycoprotein, proteolipid protein, αβ-crystallin, phosphodiesterase, transaldolase, glutamate receptor, S-100 protein), GI evaluation markers (tests for the determination of intestinal permeability, imbalances of intestinal microflora, secretory IgA, gliadin, transglutaminase, Saccharomyces cerevisiae, neutrophil cytoplasmic antigen, tropomyosin, MUC-1), immunity and chronic fatigue markers (NK cell activity, 2-5A Synthetase, RNAse L, protein kinase RNA (PKR), protein kinase C), immunological reaction to chemicals, silica, silicones, and biomarkers of oxidative stress (superoxide dismutase, catalase, glutathione peroxidase, ascorbic acid, DNA adducs, lipid peroxidation, protein adducts, apoptosis).

Without being bound to a particular theory, deficiencies in helper T cell functions, including TH1, TH2 and TH3 regulatory immune suppressor cells may contribute to defects or deficiencies seen in immune systems. For example, abnormal immune system functions accompanied by decreased energy output or negative energetic evaluations may be correlated with an excess of TH3 regulatory immune suppressor cells, and a concomitant inactivation of TH1 and TH2 T-helper cells. Restoring the proper function of the TH3 response may result in a reactivation of the TH1 response. Upon restoration of the proper TH1 response, cross-reactivity can be demonstrated to specific TH1 immune antagonists, such as viruses, bacteria, specific vaccines, parasites, metal and other foreign pathogens recognized by the TH1 system.

Identification of the underlying pathogenic or infective agent(s) that are reactive with environmental antigens will allow the design and construction of specific vaccines to the identified microbial pathogens. Treatment regimens, such as vaccines, may then be prepared using either standard vaccine protocols known to those of skill in the art. Bioelectric replications using computer generated electromagnetic signals may also be used as a treatment regimen or as a vaccine. The bioelectric replication treatment is a digital representation of the infective agent; yet as a homeopathic like substance carries none of the infective agent, but purely the digital imprint capable of stimulating an immunoglobulin response and immunity to the targeted allergen. See, e.g., U.S. Pat. No. 6,142,927, which is herein incorporated by reference in its entirety.

Included in the present invention is a library for the detection of antigens, for example environmental allergens, in biological samples. A library is defined as a set of antigens, such as environmental allergens, that react immunologically with at least some of the affected individuals. In some embodiments, it is preferable to select antigens in the library that are reactive with a large percentage of affected individuals or patients. In other embodiments it is preferable that some antigens in the library may not be completely specific. The library may be immobilized onto a solid platform, e.g. a microtiter plate, filter membrane, magnetic bead, or other immobilizing support. In other applications, the antigens in the library may be conjugated and labeled to differentiate each member of the library from the other. For example, each antigen may be labeled with a unique semi-conductor nanoparticle, such that upon binding of an IgE, IgA or other immunoglobulin molecule in a biological sample, and subsequent washing and purification, the assay will detect only that frequency of unique semi-conductor nanoparticle, and thus reveal reactivity with the specific antigen, e.g. a microbial antigen or environmental allergens.

After the identification of positively reacting antigens, for example environmental allergens, specific molecular tests can be performed to obtain the identity of infective agents responsible for the adverse reaction, such as an allergic reaction, through molecular or homeopathic assays. For example, such tests may utilize polymerase chain reaction (PCR) to identify the molecular fingerprint of each microbial infective agent. To do this, each separate compartment or area of a solid platform, for example a 96-well microtiter plate, contains a probe set to a specific microbial agent, for example E. coli or Helicobacter pylori. PCR probes to specific microbial agents are well known to those of ordinary skill in the art, and may be designed using nucleotide sequences unique or specific to each microbial agent. For example, U.S. Pat. No. 7,294,490, which is incorporated by reference herein, employs amplification means, including PCR, for detecting and identifying specific microbial agents in a biological sample. Each solid platform may contain probe sets specific to a microbial agent group, for example, a panel of bacterial agents. The PCR test may be repeated on other platforms directed specifically to other groups of microbial agents, for example, viral agents, spirochetes, bacteriophages, parasites and other microbial agents or pathogens. Each subsequent assay provides a precise DNA fingerprint of the underlying microbial infective agents, allowing identification of specific bacterial and/or viral species responsible for the allergic reaction. The results from each PCR test will be compared to the results obtained from the environmental allergen testing stage, and correlated thereto.

Quantitative real-time PCR enhanced immunoassay is a preferred means of detecting and identifying the underlying infectious agent in an individual. The methods for performing quantitative real-time PCR (QRT-PCR) are well known to practitioners. QRT-PCR uses RNA as a template prepared from a biological sample, and allows accurate and precise quantification of specific RNA sequences in the sample. QRT-PCR enhanced immunoassay takes advantage of the high sensitivity through amplification of a specific RNA sequence, for example bacterial or viral antigen, captured by a specific immunoglobulin in a patient's sample. For example, previous studies have measured viral immunoglobulin M (IgM) antibody by first capturing IgM in the patient's sample onto anti-IgM attached to a solid support substrate. See Elfaitouri et al., 2005, Clin. Diag. Lab. Immun. 12:235-241. Enteroviral antigen is then allowed to bind to the captured IgM. Any RNA bound to the IgM is released through denaturation of the complex, and amplified via QRT-PCR. Quantification of a patient's IgM specific to enteroviral antigen allows analysis of any recent exposure to an enteroviral infectious agent.

Alternatively, the specific pathogen or infectious agent may be identified through acupuncture-based or kinesiology assays, including electrochemical detection and identification of the pathogen or infectious agent, electroacupuncture detection or kinesiology-based assays. Digital response technique (DRT), which consists of an expansive series of glass vials programmed with an electrical frequency that corresponds to a particular characteristic of a pathogen or other microbial species, may also be used to detect pathogen or infectious agents. Briefly, a patient holds a vial containing the electrical frequency of a pathogen in question. As the patient touches each of the vials, a muscle reactivity test is performed at predetermined acupuncture points on the patient's body. Each vial pertaining to a debilitating condition, or causative factor, has a different frequency that is programmed by, for example, an SE5 machine. One of the main advantages of DRT is that it measures functional or useful energy rather than total energy. See U.S. Pat. No. 7,217,281, which is incorporated herein by reference in its entirety.

A galvanic skin response (GSR) test (Zyto™ Biocommunication Testing) may also be used to identify the specific pathogen or infectious agent. Briefly, GSR testing uses non-invasive digital stressor technology to determine and quantify various biomarkers, including allergens, immunoglobulins, and may detect pathogens and other microbial agents. Briefly, the patient is queried with a digital stressor that digitally simulates environmental stressors to elicit a cellular response in the patient. Changes in the galvanic skin response of the patient is then measured in response to the digital environmental stressor, resulting in an output that details the specific interaction of allergens, immunoglobulins and/or pathogens and other microbial agents that may reside in the patient. See U.S. patent application Ser. No. 11/521,417 (Pub. No. 2007/0066874), which is incorporated by reference in its entirety.

A “subject,” “individual” or “patient” is used interchangeably herein, and refers to a vertebrate, preferably a mammal, more preferably a human. Mammals include, but are not limited to, murines, simians, humans, farm animals, sport animals, and pets. Tissues, cells and their progeny of a biological entity obtained in vivo are also encompassed.

As used herein, “sample” encompasses a variety of sample types and origins, such as blood and other liquid samples of biological origin, solid tissue samples such as a biopsy specimen or tissue cultures or cells derived therefrom, and the progeny thereof. The term “sample” encompasses a clinical sample, and also includes cells in culture, cell supernatants, cell lysates, serum, plasma, biological fluid, and a pure or enriched bacterial or viral sample derived from any of these, for example, as when a sample is cultured in order to increase, enrich and substantially purify a bacterial or viral sample therefrom. The term “sample” further encompasses environmental samples, such as water or soil samples. A sample can be from a microorganism, e.g., bacteria, yeasts, viruses, viroids, molds, fungi, plants, animals, including mammals such as humans. A sample may comprise a single cell or more than a single cell. These samples can be prepared by methods known in the art such as lysing, fractionation, purification, including affinity purification, FACS, laser capture microdissection (LCM) or isopycnic centrifugation. The term “sample” can also include products of subcellular fractionation methods used to create enriched cellular or subcellular fractions, such as subcellular organelles including nuclei, mitochondria, golgi apparatus, endoplasmic reticulum, chloroplasts, heavy and light membranes and cytoplasm. Furthermore, the term “sample” may also include electromagnetically imprinted information capsules, which contain binary codes that represent digital representations of analogues. Such digital analogues may be seen by the body and the immune system as replicas of the original infective agent, but do not contain the infective agent.

Samples may be obtained from any subject by any technique known in the art. Samples derived from an animal or human can include, e.g., whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. Samples comprising whole blood or plasma are preferred. Methods of separating cells and cellular components from whole blood are well known in the art. Samples comprising biopsy tissue are not preferred, although such samples can be used to practice the present invention and may be collected by any technique known in the art.

To obtain a blood sample, any technique known in the art may be used, e.g. a syringe or other vacuum suction device. A blood sample can be optionally pre-treated or processed prior to enrichment. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent. In some embodiments, a blood sample can be combined with an agent that selectively lyses one or more cells or components in a blood sample. Whole cells, cell fragments, but preferably proteins will be isolated from the samples by any technique known in the art.

Preparation of samples are described herein and well known in the art. It is understood that a sample comprising bacteria or virus can be removed from its source (e.g., an individual, food, air, water, and other environmental samples); grown in culture, whereby the bacteria and/or virus is multiplied, enriched and/or purified (in some embodiments, substantially purified) prior to preparation of protein sample. Proteins can prepared from a whole cell extract or can be pre-fractionated based on subcellular location (e.g., membrane and cytoplasmic) or based on different physical and functional properties. In some embodiments, serum suspected of comprising bacteria and/or virus is depleted of the major serum proteins prior to analysis using digital antibodies. Methods for depleting, reducing and/or removing the major serum proteins are well known in the art. Proteins can also be extracted from the supernatant of a culture. In some embodiments, the sample is comprised of (derived from) mammalian cells (in some embodiments, vertebrate cells), such as human, murine, primate, or rodent. In some embodiments, the cell is of a non-human mammal (in some embodiments, of a non-human vertebrate).

Detection assays employing radioactive or fluorescent labels are contemplated within the present invention. The particular label or detectable moiety used and the particular assay are not critical aspects of the invention. The detectable moiety can be any material having a detectable physical or chemical property. Such detectable labels have been well developed in the field of gels, columns, and solid substrates, and in general, labels useful in such methods can be applied to the present invention. Thus, a label is any composition detectable by spectroscopic, photochemical, biochemical, immunochemical, electrical, optical or chemical means. Furthermore, it will be recognized that fluorescent labels are not to be limited to single species organic molecules, but include inorganic molecules, multi-molecular mixtures of organic and/or inorganic molecules, crystals, heteropolymers, and the like.

Useful labels in the present invention include fluorescent dyes (e.g., fluorescein isothiocyanate, Texas red, rhodamine, and the like), enzymes (e.g., LacZ, CAT, horse radish peroxidase, alkaline phosphatase, I²-galactosidase, β-galactosidase, and glucose oxidase, acetylcholinesterase and others, commonly used as detectable enzymes), quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, molecular beacons and fluorescent derivatives thereof, a luminescent material such as luminol; light scattering or plasmon resonant materials such as gold or silver particles or quantum dots; or radiolabels including ¹⁴C, I²³¹, I²⁴¹, I³¹¹, I²⁵¹, Tc99m, ³²P, ³⁵S or ³H; or spherical shells, and probes labeled with any other signal generating label known to those of skill in the art, as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland.

Semi-conductor nanocrystals such as quantum dots (i.e., Qdots) described in U.S. Pat. No. 6,207,392, are commercially available from Quantum Dot Corporation and include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same. Examples of labels can also be found in U.S. Pat. Nos. 4,695,554; 4,863,875; 4,373,932; and 4,366,241. Colloidal metals and dye particles are disclosed in U.S. Pat. Nos. 4,313,734 and 4,373,932. The preparation and use of non-metallic colloidals are disclosed in U.S. Pat. No. 4,954,452. Organic polymer latex particles for use as labels are disclosed in U.S. Pat. No. 4,252,459.

In addition to the previously mentioned labels, other labels which rely on proximity or quenching can be utilized. One such example of a proximity-dependent label is the AlphaScreen system available from PerkinElmer which allows for identification without the need for solid support. Quenching and other methods for detecting the binding of two antibodies to a single ligand have been described and are well known in the art. See, e.g., U.S. Pat. Nos. 4,199,559; 5,672,475; 5,783,453; and 6,013,457.

The label may be coupled directly or indirectly to the protein according to methods well known in the art. Methods for attaching and/or linking (either covalently or noncovalently, directly or indirectly, e.g., via a linker) label to protein are well known in the art. As indicated above, a wide variety of labels may be used, with the choice of label depending on the sensitivity required, ease of conjugation of the compound, stability requirements, available instrumentation, and disposal provisions. Non-radioactive labels are often attached by indirect means. In some embodiments, a ligand molecule (e.g., biotin) is covalently bound to a polymer. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled anti-ligands. Alternatively, any haptenic or antigenic compound can be used in combination with an antibody.

Labels can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, fluorescent green protein, and the like. Chemiluminescent compounds include luciferin, and 2,3-dihydrophthalazinediones, e.g., luminol.

Means of detecting labels are well known to those of skill in the art. Thus, for example, where the label is a radioactive label, means for detection include a scintillation counter, proximity counter (microtiter plates with scintillation fluid built in), or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDS) or photomultipliers and the like. Similarly, enzymatic labels may be detected by providing appropriate substrates for the enzyme and detecting the resulting reaction product. Finally simple colorimetric labels are often detected simply by observing the color associated with the label.

Solid or semi-solid supports suitable for immobilizing environmental allergens or microbial or pathogenic substrates, and binding or reacting biological samples are well known in the art. Examples of a solid support include: a bead (including magnetized beads), microwell plate, a protein microarray (e.g., technology owned by Zyomyx, Inc. See, e.g. U.S. Pat. No. 6,365,418). Thus, for example, CdSe—CdS core-shell nanocrystals enclosed in a silica shell can be easily derivatized for coupling to a biological molecule. Bruchez et al. (1998) Science 281: 2013-2016. Similarly, highly fluorescent quantum dots (zinc sulfide-capped cadmium selenide) have been covalently coupled to biomolecules for use in ultrasensitive biological detection. Warren and Nie (1998) Science 281: 2016-2018. Fluorescently labeled beads are commercially available from Luminex and Quantum Dot. In addition, pads, film, nanowells, or microfluid channels can also serve as a solid support. In some embodiments, reactive antibodies may be immobilized, bound or linked on a solid or semi-solid surface such as polyvinylidene difluoride, nitrocellulose, agarose, and/or polyacrylamide gel pads. Glass slides activated with aldehyde, polylysine, or a homofunctional cross-linker can also been used.

PCR—The most commonly used methods known in the art for detection of nucleic acids in a sample, for example, expression of mRNA in a biological sample include southern blotting, northern blotting and in situ hybridization (Parker & Barnes, Methods in Molecular Biology 106:247-283 (1999)); RNAse protection assays (Hod, Biotechniques 13:852-854 (1992)); and PCR-based methods, such as RACE (rapid amplification of cDNA ends) or reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridisation (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), or RNA microarrays.

Reverse Transcriptase PCR (RT-PCR): One of the most sensitive and most flexible quantitative PCR-based gene expression profiling methods is RT-PCR, which can be used to compare mRNA levels in different samples to characterize patterns of gene expression, to discriminate between closely related mRNAs, or to analyze RNA structure.

The first step is the isolation of mRNA from a biological sample. For example, the starting material can be typically total RNA isolated from blood or tissue. mRNA can also be extracted, for example, from frozen or archived fixed tissues, for example paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling and identification by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

In addition to RT-PCR, identification of microbial agents may also be obtained through amplification of genomic DNA. Amplification of unique non-coding regions, for example, operons, upstream control elements or other regions of a specific microbial infective agent may allow for specificity when designing probes sets for carrying out these assays. To accomplish, genomic DNA may be obtained using any conventional means possible. Extraction of genomic DNA is well known to those of skill in the art, and can be performed, for example, using CsCl gradient purification, or through the use of commercial kits available for the purpose of isolating genomic DNA for PCR amplification.

Some embodiments of the invention include immunoassay for the identification of nucleic acid or proteins specific to the microbial infective agent and/or environmental allergen. In immunoblotting, e.g. western blots, proteins are electrophoretically separated and then identified through binding of its antibody. An immunoassay can be a competitive binding immunoassay where an analyte competes with a labeled antigen for a limited pool of antibody molecules (e.g. radioimmunoassay, EMIT). An immunoassay can also be non-competitive, where antibody is present in excess and is labeled. As analyte antigen complex is increased, the amount of labeled antibody-antigen complex may also increase (e.g. ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the biological samples used herein. The antibodies—either used as primary or secondary ones—can be labeled with radioisotopes (e.g. 125I), fluorescent dyes (e.g. FITC) or enzymes (e.g. HRP or AP) which may catalyse fluorogenic or luminogenic reactions, as described above.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-aminohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include an ELISA assay to analyze the environmental allergens and/or microbial infective agents. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that were bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments of the invention may include fluorescence immunoassays for the identification and analysis of microbial infective agents and/or environmental allergens. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical crosslinking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify cells that express the microbial infectious agent. FACS is a specialised type of flow cytometry. It provides a method for sorting a heterogenous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate expression of the identified differentially regulated genes.

Mass spectrometry can also be used to characterize expression of microbial infective agents from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyser. In the second, proteins are enzymatically digested into smaller peptides using an agent such as trypsin or pepsin. Other proteolytic digest agents are also used. The collection of peptide products are then introduced to the mass analyser. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g. non-radioactive) heavier isotopes of carbon (C13) or nitrogen (N15) are incorporated into one sample while the other one is labelled with corresponding light isotopes (e.g. C12 and N14). The two samples are mixed before the analysis. Peptides derived from the different samples can be distinguished due to their mass difference. The ratio of their peak intensities corresponds to the relative abundance ratio of the peptides (and proteins). The methods for isotope labelling are SILAC (stable isotope labelling with amino acids in cell culture), trypsin-catalyzed O18 labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilises the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g. using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

Vaccines—In another embodiment of the invention, the immunoreactive polypeptides (including allergens) or structural analogs of epitopes, may be prepared by conventional means into vaccines. Vaccines may be prepared from one or more immunogenic polypeptides isolated from the microbial infective agents. If made by recombinant technology, these polypeptides are suitably expressed in a variety of host cells (e.g., bacteria, yeast, insect, or mammalian cells). Alternatively, the antigens may be isolated from microbial preparations or prepared synthetically if the amino acid sequence is known.

The preparation of vaccines which contain, as active ingredients, an immunogenic polypeptide or structural analog having epitopes is known to one skilled in the art. Typically, such vaccines are prepared as injectable liquid solutions or suspensions. Solid forms suitable for solution in, or suspension in a liquid prior to injection are also prepared. The preparation may also be emulsified, or the protein encapsulated in liposomes.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium stearate,-sodium saccharine, cellulose, magnesiumcarbonate, and the like. These compositions take the form of solutions, suspensions, tablets, pills, capsules, sustained release formulations, oral sprays, transdermal patch or powders and contain 10%-95% of active ingredient, preferably 25%-70%.

Vaccines within the present invention are administered in a manner compatible with the dosage formulation, and in such amount as will be prophylactically and/or therapeutically effective. The quantity to be administered, which is generally in the range of about 5 micrograms to about 250 micrograms of antigen per dose, depends on the subject to be treated, capacity of the subject's immune system to synthesize antibodies, and the degree of protection desired. Precise amounts of active ingredient required to be administered may depend on the judgment of the practitioner and may be peculiar to each subject.

The vaccine may be given in a single dose schedule, or preferably in a multiple dose schedule. A multiple dose schedule is one in which a primary course of vaccination may be with 1-10 separate doses, followed by other doses given at subsequent time intervals required to maintain and or reenforce the immune response, for example, at 1-4 months for a second dose, and if needed, a subsequent dose(s) after several months. The dosage regimen is also, at least in part, determined by the need of the individual and be dependent upon the judgment of the practitioner.

Alternatively, the vaccine may be prepared using bioelectric replications from computer-generated electromagnetic signals. One example of the use of electromagnetic signals for treatment of disease states in seen in U.S. Pat. No. 6,142,927, which is fully incorporated by reference therein. A product capsule, which represents an identified bacteria, virus, microbe and/or an infective agent mathematical replica, may be held in either a programmable solution by a series of binary numbers or a computer memory. Such information may be transmitted via several methods; e.g. radio frequency, antenna, and other transmission means. This method may also enhance the strength of such solution by the number of binary sequences. The information of said infective agents may be represented by such binary memory capsules, thus carrying only the coded infective information and none of the infectivity of the infective sample. Such method then permits a non-infective replica to be safely used by the body as a mirage of the original, which can be rotated either cis or trans to stimulate an immune response as desired.

The administration of a variety of agents or processes may act to alter the resistance value in an individual. One means of altering resistance values include the application of electromagnetic signals to the individual. Such electromagnetic signals can be generated through the measurement of resistance values upon application of a defined electrical and magnetic energy to an individual. U.S. Pat. No. 6,142,297 describes one means of defining and administering an electrical and magnetic signal, including the use of a computer generated binary value to define a resonant signal. A vaccine can be produced based on the electromagnetic binary value necessary to produce an alteration in resistance value, and administered to a patient to treat an underlying microbial infective agent.

Treatment—Also contemplated within the present invention is the treatment of an individual diagnosed with an allergic reaction. This method includes the steps of: 1) diagnosing the traditional allergen responsible; 2) determining the correlative underlying infectious agent; and 3) treating said patient with a medicament or system that may reduce or eliminate the infectious agent from the patient. Identification of the causative microbial infectious agent may be through the methods disclosed herein. Alternatively, treatment may occur based on questionnaires presented by said individual afflicted with the allergic reaction. The questionnaire may present a series of questions regarding the environmental factors or stimuli that may affect said patient. The questionnaire may also reveal previous tests performed, and results of the tests. For example, a skin prick test may have already revealed said patient's sensitivity to cat dander or pollen. Results from the questionnaire may be correlated with knowledge obtained previously on correlative tests performed to determine which underlying microbial infectious agent is involved in said patient's allergic reaction.

After identification of the causative microbial agent, conventional treatment with medicaments towards the reduction or eradication of the identified microbial agents are also contemplated. Such medicaments may include, but are not limited to, antibiotics, including pencillins, cephalosporins, tetracyclines, erythromycin, beta-lactams, sulfa drugs, including sulfonamides, and antifungal antibiotics, aminoglycosides, ansamycins, carbacepham, carbapenems, macrolides, quinolones, small molecule inhibitors polypeptides, anti-viral agents, including nucleoside analogues and interferon therapies, such as acyclovir, gancyclovir, zidovudine, lamivudine, ribavirin, amantidine and protease inhibitors.

Alternatively, phage therapy, interleukin-interferon manipulation, or any other means of reducing or eliminating the underlying infectious agent, may be used to reduce or eliminate the underlying microbial infective agent. Phage therapy administers a viral-bacterial match, whereby a practitioner identifies a virus that is capable of eliminating the identified bacteria in vivo. By selectively administering the specific virus, targeting of the identified bacterial species can be accomplished by invasion of the virus of the bacteria and elimination in vivo.

### EXAMPLES

### Example 1

### Identification and Treatment of Allergy Subjects

Patients suitable for allergy treatment may range in age from toddlers and children to older adults in their 60's and 70's. Most patients appear after they have seen a number of both traditional physicians and sometimes alternative physicians without clinical result. Types of cases include autism, lyme disease, chronic fatigue, skin issues, chronic allergies, chronic infections and emotional/psychiatric disorders.

Examination is performed to determine if an altered autonomic nervous system response is present. Such examinations include a Heart Rate Variability Test, as well as a Bio Impedance Analysis, which may show decreased intra cellular hydration and increased extracellular hydration, as well as changes in cellular phase angle. Other specific laboratory tests include circadian cortisol release, as well as decreased secretory IgA markers, food allergies and specific biomarkers for various diseases; Crohn's disease, type I diabetes, multiple sclerosis, parasites and other specific and known laboratory markers. In addition to the laboratory tests, energetic evaluations are also performed. Decreased energy production in a patient is often accompanied by abnormalities of immune function resulting in an inability to maintain an immune response under static or stressed conditions. This condition is marked by an excess of TH3 regulatory immune suppressor cells and an inactivation of TH1 and TH2 T-Helper Cells.

Restoring the proper function of the TH3 results in a reactivation of the TH1 response at which time a cross reaction can be demonstrated to specific TH1 immune antagonists; virus, bacteria, specific vaccines, parasites, metal, and other antigens. For example, upon restoration of the TH1 response, the immune system will cross react to bacteria as the immune stimulant needing immediate attention. Using a technique to cross match or manually biopan the patient's responses to multiple bacteria samples, a candidate for further testing is then selected. The positive response to the bacterial sample is then cross tested to identify the type of reactivity, either allergic or infective. Further testing of the selected bacteria along with a neutralizer may show reactivity to viral samples. This cross reactivity of the neutralized bacterial sample to the target virus is also done in a manual biopanning method until the target virus or pathogen and/or serotype is identified. Other techniques include short cycling peptidic immune response to induce amino acid sequence modifications and resultant stimulation of the TH1 or TH2 response. Other assays for determining relationships include amino acid sequence determination, structural pattern recognition, automated biopanning, B.L.A.S.T. searches and real time PCR.

Once the virus or pathogen is identified, a bioelectrical inversion of the virus or pathogen is tested to determine efficacy in the elimination of patient test responses. Final treatment is with a bioelectrical or bio resonant inversion footprint of the causative virus, as what may be thought of as a homeopathically bacteriophage i.e. HOMEOPHAGE™ (homeopathic bacteriophage treatment). In an allergy, protocol the allergen is cross tested to bacterial samples in a manual biopanning manner. The resultant matching bacteria is then further cross matched to the virus for Homeophage™ preparation.

### Example 2

### Homeophage™ Preparation

Bacteriophages are specialized viruses which invade, infect and kill bacteria in the environment. Once bacterial or other microbial species are identified as the pathogenic or infectious agent source in a patient, a Homeophage™ treatment regimen is determined through the creation from a bioelectric footprint of a virus which is specifically cross matched to a targeted bacterium/microbe. The Homeophage™ treatment targets, for example, an amino acid sequence of the unresolved immune reaction from the patient to a microbe or bacterium. The Homeophage™ treatment, therefore, contains no virus material but only the homeopathic or digital imprint of the specific bacteriophage virus needed. The bioelectric cis or trans version of the virus stimulates an immune reaction and only attacks specific bacteria. With removal of these bacteria from the immunological wanted poster the immune system can reset and is no longer aggressively in search of look-a-like proteins of either self, environment or diet.

### Example 3

### Identification of Correlative Treatment of Patients

A patient presents with allergy symptoms, including adverse reaction to dietary components (gluten). A blood sample is taken, and immunoglobulin-antigen reactive complexes are detected using RAST. The patient is confirmed for allergies to gluten. The correlative microbial pathogen is determined using the methods of Example 1. The underlying microbial pathogen is determined using the methods of Example 1. The underlying microbial pathogen is determined to be an unresolved strep infection. This same strep infection is found to be responsible for other localized or systemic disease and/or symptoms, including Obsessive Compulsive Disorders.

A Homeophage™ to the underlying strep infection is developed by synthesizing the bioelectrical footprint which has the most specific predilection for the unresolved infective material. The patient is treated with the cis or trans bioelectric virus which has been imprinted into a structured carrier solution until the symptoms of both the gluten allergy and behavioral disorder are resolved.

### REFERENCES

1. Scott et al, Searching for peptide ligands with an epitope library, Science 249(4967):386-390 (July 1990)

2. Muller et al, Part 1. New Principles of Autoimmunity Human Proteinase 4(PR3) and its Binding Molecules, Ann. NY Adac. Sci. 1109:94-92 (2007)

3. Asthma and Allergy—The Revenge of the Viral Nerd?

4. Imani, Farhad, PhD. Immunology Group, Mechanisms of Inflammation, NIEHS (July 2007)

5. Toll-like Receptor Regulation of Allergic Pulmonary Inflammation, NIEHS (June 2007)

6. Pauwels et al, IgE Antibodies to Bacteria in Patients with Bronchial Asthma, Allergy 35(8):665-669 (December 1980)

7. Nandi et al, Topological mimicry and epitope duplication in the guanylyl cyclase C receptor, Protein Sci. 7(10) (October 1998)

8. Malling, H. J., Baterial vaccines:anything but placebo, Allergy 55(3):214-218 (2000)

9. Guameri et al, Identification of Potentially Cross-Reactive Peanut-Lupine Proteins by Computer-Assisted Search for Amino Acid Sequence Homology, Allergy and Immunology 138(4) (2005)

10. Czerkinsky et al, IgA Antibody-Producing Cells in Peripheral Blood after Antigen Ingetion:Evidence for a Common Mucosal Immune System in Humans, PNAS 84(8):2449-2453 (April 1987)

11. Basagana et al, Allergy to human seminal fluid:Cross-reactivity with dog dander, J Allergy Clin Immunol, (November 2007)

12. Holgate, Stephen T, Science, medicine, and the future; Allergic disorders, BMJ 320:231-234 (January 2000)

13. Meitin et al, Influenza immunization: intranasal live vaccinia recombinant contrasted with parenteral inactivated vaccine, Vaccine 9(10):751-6 (October 1991)

14. Moldoveanu et al, Human immune responses to influenza virus vaccines administered by systemic or mucosal routes, Vaccine 13(11):1006-12 (August 1995)

15. Weisz-Carrington et al, Organ and Isotype Distribution of Plasma Cells Producing Specific Antibody after Oral Immunization: Evidence for A Generalized Secretory Immune System, Journal of Immunology 123:1705-1708 (1979)

16. Wines et al, IgA receptors in health and disease, Tissue Antigens 68(2): 103-114 (August 2006)

17. Elfaitouri et al., Quantitative PCR-enhanced immunoassay for measurement of enteroviral immunoglobulin M antibody and diagnosis of aseptic meningitis, Clin. Diagnostic Lab. Immun. 12:235-241 (February 2005).

While preferred embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of correlating an underlying pathogen with an antigen, the method comprising the steps of:
(a) obtaining a library of purified and isolated antigens;
(b) providing a biological sample from an individual afflicted with an adverse reaction related to an immune disorder;
(c) reacting the biological sample from the individual with each of the antigen library and the pathogen library;
(d) detecting a reactive complex between at least one purified and isolated antigen and the biological sample;
(e) detecting the presence of at least one pathogen in the biological sample; and
(f) identifying the antigen(s) reacting with said biological sample and pathogen(s) in said biological sample,
wherein the identified antigen mediates the adverse reaction in the individual, and the detected pathogen(s) is an underlying pathogen(s) in the antigen-mediated adverse reaction in the subject.

2. The method of claim 1, wherein the reactive complex between at least one purified and isolated antigen and the biological sample is an immunoglobulin antigen complex.

3. The method of claim 2, wherein the immunoglobulin is IgG, IgM, IgA or IgE.

4. The method of claim 2, wherein the assay to identify an immunoglobulin-antigen complex is RAST.

5. The method of claim 1, wherein the pathogen is a bacteria, a virus, a bacteriophage, a spirochete, or a parasite.

6. The method of claim 1, wherein the pathogen is detected via PCR.

7. The method of claim 1, wherein the pathogen is detected via kinesiology, energetic testing, digital response technique (DRT), electrochemical detection, electroacupuncture, galvanic skin response (GSR) or acupuncture point testing.

8. The method of claim 1, wherein said immune disorder is an allergy, a chronic illness, chronic fatigue, fibromyalgia, Lyme's disease, an autoimmune disorder, a skin condition, hair loss, asthma, polycystic liver disease, polycystic kidney disease, autism, cancer, mental illness or combinations thereof.

9. A method of correlating and identifying an underlying pathogen responsible for an allergic reaction, the method comprising the steps of:
(a) providing a biological sample from an individual afflicted with an allergic reaction;
(b) reacting the biological sample with a library of purified and isolated allergens;
(c) detecting the presence of an allergen antigen-immunoglobulin complex for at least one of the allergens in the library;
(d) detecting the presence of at least one pathogen in the biological sample; and
(e) identifying the detected allergen in the antigen-immunoglobulin complex and the detected pathogen;
wherein the detected pathogen is correlated with the detected allergen, and the detected pathogen is an underlying pathogen in the allergic reaction.

10. The method of claim 9, wherein the immunoglobulin is IgG, IgM, IgA or IgE.

11. The method of claim 9, wherein the assay to identify the allergen is RAST.

12. The method of claim 9, wherein the pathogen is a bacteria, a virus, a bacteriophage, a spirochete, or a parasite.

13. The method of claim 9, wherein the assay to identify the pathogen is PCR.

14. The method of claim 9, wherein the assay to identify the pathogen is kinesiology, energetic testing, digital response technique (DRT), electrochemical detection, electroacupuncture, galvanic skin response (GSR) or acupuncture point testing.

15. A diagnostic kit comprising a library of allergen antigens, a means for detecting immunological complexes formed between at least one allergen antigen in the library and an immunoglobulin in a biological sample, a means of detecting a pathogen in said biological sample, and a means of correlating the formation of complexes between an antigen and an immunoglobulin in the biological sample and said detected pathogen in the biological sample.

16. The diagnostic kit of claim 15, wherein the immunoglobulin detected is IgG, IgM, IgA or IgE.

17. The method of claim 15, wherein the means for identifying the formation of complexes between an allergen antigen and an immunoglobulin is RAST.

18. The method of claim 15, wherein the pathogen detected is a bacteria, a virus, a bacteriophage, a spirochete, or a parasite.

19. The method of claim 15, wherein the means for identifying the pathogen in the biological sample is PCR.

20. The method of claim 15, wherein the means for identifying the pathogen in the biological sample is kinesiology, energetic testing, digital response technique (DRT), electrochemical detection, electroacupuncture, galvanic skin response (GSR) or acupuncture point testing.

21. A method for treatment of an individual afflicted with an allergic reaction, the method comprising the steps of:
(a) providing a biological sample from an individual afflicted with an allergic reaction;
(b) reacting the biological sample with a library of purified and isolated allergens;
(c) detecting the presence of an allergen antigen-immunoglobulin complex for at least one of the allergens in the library;
(d) identifying the allergen in the antigen-immunoglobulin complex;
(e) identifying a pathogen that correlates with the identified allergen according to the method of claim 9; and
(f) treating said individual with an agent that inhibits the identified pathogen agent.

22. The method of claim 21, wherein the assay for detecting the allergen-antigen immunoglobulin complex is RAST.

23. The method of claim 21, wherein the immunoglobulin antibody is IgG, IgM, IgA or IgE.

24. The method of claim 21, wherein the individual is treated with an anti-microbial agent.

25. The method of claim 24, wherein the anti-microbial agent is an antibiotics, a penicillin, a cephalosporin, a tetracycline, an erythromycin, a beta-lactam, a sulfa drug, a sulfonamide, an antifungal antibiotic, an aminoglycoside, an ansamycin, a carbacepham, a carbapenem, a macrolide, aquinolone, a small molecule inhibitor, a polypeptide, an anti-viral agent, a nucleoside analog, an interferon, acyclovir, gancyclovir, zidovudine, lamivudine, ribavirin, amantidine, a protease inhibitor, or a mixture thereof.

26. The method of claim 24, wherein the individual is treated with phage therapy, interleukin manipulation, interferon therapy, or a combination thereof.

27. A method for treatment of an individual afflicted with an allergic reaction, the method comprising the steps of:
(a) identifying an allergen which mediates the allergic reaction through a questionnaire completed by the individual;
(b) identifying a pathogen agent that correlates with the identified allergen as in claim 1; and
(c) treating said individual with an agent that inhibits the identified pathogen agent.

28. The method of claim 27, wherein the anti-microbial agent is an antibiotics, a penicillin, a cephalosporin, a tetracycline, an erythromycin, a beta-lactam, a sulfa drug, a sulfonamide, an antifungal antibiotic, an aminoglycoside, an ansamycin, a carbacepham, a carbapenem, a macrolide, aquinolone, a small molecule inhibitor, a polypeptide, an anti-viral agent, a nucleoside analog, an interferon, acyclovir, gancyclovir, zidovudine, lamivudine, ribavirin, amantidine, a protease inhibitor, or a mixture thereof.

29. The method of claim 27, wherein the individual is treated with phage therapy interleukin manipulation, interferon therapy, or a combination thereof.

30. A method for treatment of an individual afflicted with an immune disorder, the method comprising the steps of: providing a biological sample from an individual afflicted with an allergic reaction;
(a) reacting the biological sample with a library of purified and isolated antigens;
(b) detecting the presence of an antigen-immunoglobulin complex for at least one of the antigens in the library;
(c) identifying the antigen in the antigen-immunoglobulin complex;
(d) identifying a pathogen that correlates with the identified antigen according to the method in claim 1; and
(e) treating said individual with an agent that inhibits the identified pathogen agent.

31. The method of claim 30, wherein the immunoglobulin antibody is IgG, IgM, IgA or IgE.

32. The method of claim 30, wherein the individual is treated with an anti-microbial agent.

33. The method of claim 32, wherein the anti-microbial agent is an antibiotics, a penicillin, a cephalosporin, a tetracycline, an erythromycin, a beta-lactam, a sulfa drug, a sulfonamide, an antifungal antibiotic, an aminoglycoside, an ansamycin, a carbacepham, a carbapenem, a macrolide, aquinolone, a small molecule inhibitor, a polypeptide, an anti-viral agent, a nucleoside analog, an interferon, acyclovir, gancyclovir, zidovudine, lamivudine, ribavirin, amantidine, a protease inhibitor, or a mixture thereof.

34. The method of claim 30, wherein the individual is treated with phage therapy interleukin manipulation, interferon therapy, or a combination thereof.

35. The method of claim 30, wherein the immune disorder is an allergy, a chronic illness, chronic fatigue, fibromyalgia, Lyme's disease, an autoimmune disorder, a skin condition, hair loss, asthma, polycystic liver disease, polycystic kidney disease, autism, cancer, mental illness or combinations thereof.
